SEQUENCE LISTING (9 sequences)

SEQ ID NO: 1 - protein, 709 aa, Homo sapiens
MATAGGGSGADPGSRGLLRLLSFCVLLAGLCRGNSVERKIYIPLNKTAPCVRLLNATHQIGCQSSISGDTGVIHVVEKEEDLQWVLTDGPNPPYMVLLESKHFTRDLMEKLKGRTSRIAGLAVSLTKPSPASGFSPSVQCPNDGFGVYSNSYGPEFAHCREIQWNSLGNGLAYEDFSFPIFLLEDENETKVIKQCYQDHNLSQNGSAPTFPLCAMQLFSHMHAVISTATCMRRSSIQSTFSINPEIVCDPLSDYNVWSMLKPINTTGTLKPDDRVVVAATRLDSRSFFWNVAPGAESAVASFVTQLAAAEALQKAPDVTTLPRNVMFVFFQGETFDYIGSSRMVYDMEKGKFPVQLENVDSFVELGQVALRTSLELWMHTDPVSQKNESVRNQVEDLLATLEKSGAGVPAVILRRPNQSQPLPPSSLQRFLRARNISGVVLADHSGAFHNKYYQSIYDTAENINVSYPEWLSPEEDLNFVTDTAKALADVATVLGRALYELAGGTNFSDTVQADPQTVTRLLYGFLIKANNSWFQSILRQDLRSYLGDGPLQHYIAVSSPTNTTYVVQYALANLTGTVVNLTREQCQDPSKVPSENKDLYEYSWVQGPLHSNETDRLPRCVRSTARLARALSPAFELSQWSSTEYSTWTESRWKDIRARIFLIASKELELITLTVGFGILIFSLIVTYCINAKADVLFIAPREPGAVSY

SEQ ID NO: 2 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - protein, 119 aa, Artificial Sequence
  gamma-secretase binding antibody heavy chain variable region (VH) sequence, 10C5 VH
QVTLKESGPGMLQPSKTLSLTCSFSGFSLRSSGMVVSWIRQPSGKSLEWLAAIDWDGDKYYNPSLKSRLTVSKDTSNTQVFLKITSVDIADTATYYCARIYYGYYFDYWGQGVMVTVSS

SEQ ID NO: 4 - protein, 115 aa, Artificial Sequence
  gamma-secretase binding antibody light chain variable region (VL) sequence, 10C5 VL
DIVMTQSPSSLVVSAGETVTINCKSSQSLLSSGNQKNYLAWYQQKPGQSPKLLIYWASTRQSGVPDRFIGSGSGTDFTLTISSVQAEDLAIYYCQQYFENLPWTFGGGTKLELKR

SEQ ID NO: 5 - protein, 120 aa, Artificial Sequence
  gamma-secretase binding antibody VH sequence, 1E2 VH
EVQLVESGGGLVQPGRSMKLSCAASGFTFSNYYMAWVRQAPTKGLEWVASISTGGGNTYYRDSVKGRFTISRDNAKSTLYLQMDSLRSEDTATYYCARHRFGVPHYFDYWGQGVMVTVSS

SEQ ID NO: 6 - protein, 113 aa, Artificial Sequence
  gamma-secretase binding antibody VL sequence, 1E2 VL
DIVMTQTPSSQAVSAGEKVTMSCKSSQSLLYSENKKNYLAWYQQKPGQSPKLLIYWASTRESGVPDRFIGSGSGTDFTLTISSVQAEDLAVYYCQQYYNFNTFGAGTKLELKR

SEQ ID NO: 7 - protein, 112 aa, Artificial Sequence
  gamma-secretase binding antibody VH sequence, 10C11 VH
QVQLKESGPGLVQPSETLSLTCTVSGFSLTSYSVSWVRQPSGKGPEWMGRMWYDGDTAYNSALKSRLSISRDTSKNQVFLKMNSLQTDDTGTYYCTRDPWDWGQGVMVTVSS

SEQ ID NO: 8 - protein, 113 aa, Artificial Sequence
  gamma-secretase binding antibody VL sequence, 10C11 VL
DVVLTQTPSTLSATIGQSASISCRSSQSLLDSSGNTFLYWYLQRPGQSPQLLIYLVSILGSGVPNRFSGSGSGTDFTLKISGVEAEDLGVYYCMQATHAPLTFGSGTKLEIKR

SEQ ID NO: 9 - protein, 20 aa, Artificial Sequence
  2A-like cleavage site sequence
RAEGRGSLLTCGDVEENPGP

(19) United States
(12) Patent Application Publication
(10) Pub. No.: US 2022/0242965 A1
(43) Pub. Date: Aug. 4, 2022

(54) ENGINEERED T-CELLS CO-EXPRESSING AN ANTI-BCMA CAR AND AN ANTI-ECTOENZYME ANTIBODY AND THEIR USE IN THE TREATMENT OF CANCER

(71) Applicant: AUTOLUS LIMITED, London (GB)

(72) Inventors: Martin Pulé, London (GB); Shaun Cordoba, London (GB); Simon Thomas, London (GB); Shimobi Onuoha, London (GB); Reyisa Bughda, London (GB); Mathieu Ferrari, London (GB); Zulaikha Akbar, London (GB)

(21) Appl. No.: 17/607,720

(22) PCT Filed: May 1, 2020
(86) PCT No.: PCT/GB2020/051090

§ 371 Date: Oct. 29, 2021

(30) Foreign Application Priority Data
May 2, 2019 (GB) 1906202.5

(51) Int. Cl. C07K 16/28; C07K 14/725; C12N 5/0783; C12N 9/88

(52) U.S. Cl. CPC C07K 16/2896; C07K 14/7051; C07K 16/2878; C12N 5/0636; C12N 9/88; C12N 2510/00; C07K 2317/622; C07K 2317/626; C07K 2317/76; C07K 2319/03; C07K 2319/33; C12Y 402/01011

(57) ABSTRACT

The present invention provides a cell which expresses a chimeric antigen receptor (CAR) or engineered T-cell receptor (TCR) and secretes an agent which blocks or reduces the activity of an ectoenzyme. The cells may be used in adoptive immunotherapy approaches for the treatment of diseases such as cancer.

### FIELD OF THE INVENTION

The present invention relates to cell which expresses a chimeric antigen receptor (CAR) or engineered T-cell receptor (TCR). The cell secretes an agent which bocks or reduces the activity of an enzyme external to the cell i.e. an ectoenzyme.

### BACKGROUND TO THE INVENTION

Multiple Myeloma

Multiple Myeloma (myeloma) is a bone-marrow malignancy of plasma cells. Collections of abnormal plasma cells accumulate in the bone marrow, where they interfere with the production of normal blood cells. Myeloma is the second most common hematological malignancy in the U.S. (after non-Hodgkin lymphoma), and constitutes 13% of haematologic malignancies and 1% of all cancers. The disease is burdensome in terms of suffering as well as medical expenditure since it causes pathological fractures, susceptibility to infection, renal and then bone-marrow failure before death.

Unlike many lymphomas, myeloma is currently incurable. Standard chemotherapy agents used in lymphoma are largely ineffective for myeloma. In addition, since CD20 expression is lost in plasma cells, Rituximab cannot be used against this disease. New agents such as Bortezamib and Lenolidomide are partially effective, but fail to lead to long-lasting remissions.

There is thus a need for alternative agents for the treatment of myeloma which have increased efficacy and improved long-term effects.

Chimeric Antigen Receptors (CARs)

Chimeric antigen receptors are proteins which, in their usual format, graft the specificity of a monoclonal antibody (mAb) to the effector function of a T-cell. CARs are typically type I transmembrane domain protein with an antigen recognizing amino terminus, a spacer, a transmembrane domain all connected to a compound endodomain which transmits T-cell survival and activation signals.

The most common form of these molecules uses single-chain variable fragments (scFv) derived from monoclonal antibodies to recognize a target antigen. The scFv is fused via a spacer and a transmembrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Despite considerable clinical success in refractory B cell malignancies, CAR T cells have not resulted in durable responses in multiple myeloma and have had limited efficacy in other cancers.

Strategies to augment CAR-T cell activity are therefore needed.

B-Cell Maturation Antigen (BCMA)

BCMA is a transmembrane protein that is preferentially expressed in mature lymphocytes, i.e. memory B cells, plasmablasts and bone marrow plasma cells. BCMA is also expressed on multiple myeloma cells.

Carpenter et al (2013, Clin Cancer Res 19(8) 2048-60) describe a CAR which incorporates BCMA and demonstrate that T cells transduced to express the anti-BCMA CAR are capable of specifically killing myeloma cells from a plasmacytoma of a myeloma patient.

Although CAR approaches using anti-BCMA antibodies show promise, a particular consideration when targeting this antigen is the particularly low density of BCMA on myeloma cells, in comparison for instance with CD19 on a lymphoma cell.

There is a hierarchy of CAR T-cell activation from killing, to cytokine release to proliferation. A CAR T-cell may kill a target cell with low density antigen but fail to fully activate.

There is therefore a need for alternative CAR T-cell approaches for targeting low-density targets such as BCMA.

### DESCRIPTION OF THE FIGURES

FIG. 1: Expression of BCMA in Multiple Myeloma patients.

FIG. 2: Schematic diagram illustrating diabody-based ectoenzyme blocking to augment CAR T cell activity. A) While BCMA CARs show promise in myeloma, challenges are low BCMA tumour expression levels and a high level of soluble BCMA which acts as a decoy for anti-BCMA CAR-T cell binding. BCMA is cleaved from the cell surface by gamma secretase (GST). B) Tumour killing can be augmented by engineering a CAR to secrete a GST binding agent such as a bi-specific dAb or diabody directly into the tumour microenvironment. This increases tumour BCMA and reduces soluble BCMA levels without the systemic toxicity of GST blocking drugs.

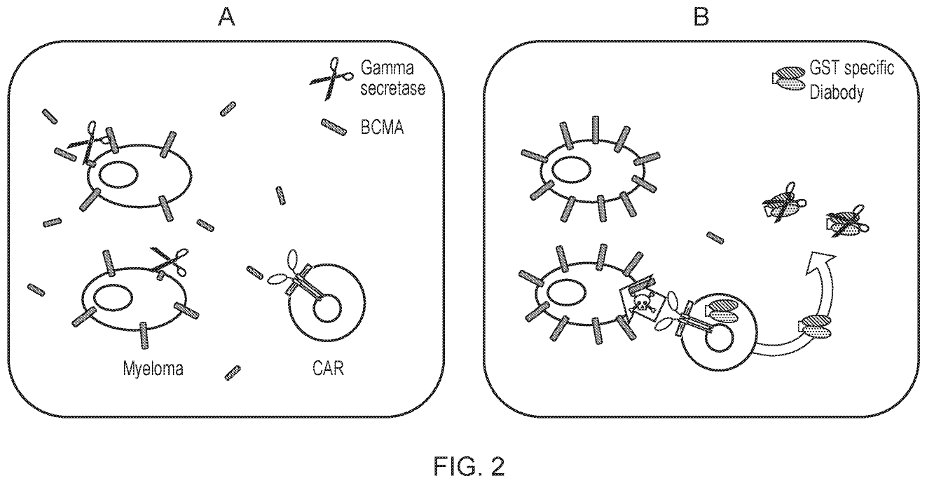

FIG. 3: Diagram illustrating non-antibody scaffold proteins.

FIG. 4: Schematic illustration of the gamma secretase complex, which is formed by the sequential assembly of APH1, nicastrin, presenilin (PS), and PEN-2.

FIG. 5: Chemical structure of DAPT.

FIG. 6: Presenilin-KLH post immunisation seroconversion ELISA following reboost

FIG. 7: The effect of α-PS-1 hybridoma single clone supernatant on A549 cells

FIG. 8: ELISA to show binding of supernatant from various clones to human nicastrin

### SUMMARY OF ASPECTS OF THE INVENTION

The present inventors have engineered chimeric antigen receptor (CAR) or engineered T-cell receptor (TCR) expressing cells to secrete an agent which blocks or reduces the activity of an ectoenzyme.

As mentioned above, although BCMA is an attractive target for CAR- and TCR-based approaches for the treatment of multiple myeloma, the low level of expression of BCMA on tumour cells is a challenge. Moreover, there is also a high level of soluble BCMA in the tumour microenvironment which acts as a decoy for anti-BCMA CAR- or TCR-binding. BCMA is cleaved from the cell surface by gamma secretase (GST). The present inventors have found that killing of BCMA expressing cells can be augmented by engineering the CAR- or TCR-expressing cell to secrete a GST-blocking factor, such as a bispecific domain antibody (dAb). The secreted factor reduces GST-mediated cleavage of BCMA from the cell surface, which has the double-beneficial effect of increasing the tumour expression level of BCMA and reducing the level of soluble BCMA in the tumour microenvironment.

The technology of the present invention has many applications. Blocking ectoenzymes can be used to reduce factors in the microenvironment which are either required by a tumour cell for survival, proliferation, metastasis or chemoresistance, or detrimental to the survival, proliferation or activity of the CAR- or TCR-expressing cell. As ectoenzymes are ubiquitous, systemic inhibition of these enzymes would be likely to be prohibitively toxic. However, the present invention provides a mechanism for the local secretion of an ectoenzyme blocker avoiding such systemic toxicities.

Thus, in a first aspect the present invention provides a cell which expresses a chimeric antigen receptor (CAR) or engineered T-cell receptor (TCR) and secretes an agent which blocks or reduces the activity of an ectoenzyme.

The agent may, for example, be or comprise an antibody or fragment thereof, a single-domain antibody, a diabody or a non-antibody scaffold polypeptide.

The agent may be bivalent. The agent may target two separate epitopes on the ectoenzyme. The agent may target two enzymatic domains on the ectoenzyme.

The ectoenzyme may be secreted by or expressed on the outer surface of a tumour cell.

Blocking or reducing the activity of the ectoenzyme may directly or indirectly affect the target antigen for the CAR or engineered TCR.

The ectoenzyme may cleave the target antigen from the target cell surface (i.e. cleavage of the target antigen from the target cell surface is one of the normal activities of the ectoenzyme in vivo in the absence of inhibition by the agent).

Blocking or reducing the activity of the ectoenzyme may increase the level of target antigen on the target cell.

The ectoenzyme may cleave transmembrane protein(s) (i.e. cleavage of one or more transmembrane protein(s) is one of the normal activities of the ectoenzyme in vivo in the absence of inhibition by the agent).

The target antigen for the CAR or engineered TCR may be B cell maturation antigen (BCMA). The ectoenzyme may be gamma secretase (GST).

Blocking or reducing the activity of the ectoenzyme may directly or indirectly reduce immune suppressing factors.

The immune suppressing factor may, for example, be adenosine. The ectoenzyme may be selected from one of the following ectonucleotidases: CD39 and CD73.

Blocking or reducing the activity of the ectoenzyme may directly or indirectly reduce immune suppressing cell type(s) such as dendritic cells. The ectoenzyme may be the glycolytic enzyme ENO1.

In a second aspect, the present invention provides a nucleic acid construct which comprises: a first polynucleotide which encodes a chimeric antigen receptor (CAR) or engineered transgenic T-cell receptor (TCR); and a second polynucleotide which encodes an agent which blocks or reduces the activity of an ectoenzyme.

The first and second polynucleotides may be separated by a co-expression site.

In a third aspect, the present invention provides a vector comprising a nucleic acid construct according to the second aspect of the invention.

In a fourth aspect, the present invention provides a kit of polynucleotides which comprises: a first polynucleotide which encodes a chimeric antigen receptor (CAR) or engineered transgenic T-cell receptor (TCR); and a second polynucleotide which encodes an agent which blocks or reduces the activity of an ectoenzyme.

In a fifth aspect, the present invention provides a kit of vectors which comprises: (i) a first vector comprising a polynucleotide which encodes a chimeric antigen receptor (CAR) or engineered transgenic T-cell receptor (TCR); and (ii) a second vector comprising a polynucleotide which encodes an agent which blocks or reduces the activity of an ectoenzyme.

In a sixth aspect, the present invention provides a pharmaceutical composition which comprises a plurality of cells according to the first aspect of the invention.

In a seventh aspect, the present invention provides a pharmaceutical composition according to the sixth aspect of the invention, for use in treating cancer.

In an eighth aspect, the present invention provides a method for treating cancer, which comprises the step of administering a pharmaceutical composition according to the sixth aspect of the invention to a subject in need thereof.

The method may comprise the following steps:
(i) isolation of a cell containing sample;
(ii) introducing a nucleic acid construct, a vector, a kit of polynucleotides, or a kit of vectors of the invention into the cell ex vivo; and
(iii) administering the cells from (ii) to a subject.

In a ninth aspect, the present invention provides the use of a cell according to the first aspect of the invention in the manufacture of a medicament for the treatment of cancer.

In a tenth aspect, the present invention provides a method for making a cell according to the first aspect of the invention, which comprises the step of introducing a nucleic acid construct, a vector, a kit of polynucleotides, or a kit of vectors of the invention into a cell ex vivo.

### DETAILED DESCRIPTION

Chimeric Antigen Receptors

The present invention relates to a cell which expresses a chimeric antigen receptor (CAR) or engineered T-cell receptor (TCR).

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus, the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain-spacer domain-transmembrane domain-intracellular signaling domain (endodomain).

Antigen Binding Domain

The antigen binding domain is the portion of the chimeric receptor which recognizes antigen. In a classical CAR, the antigen-binding domain comprises: a single-chain variable fragment (scFv) derived from a monoclonal antibody.

CARs have also been described in which the antigen-binding domain is based on a ligand for the target antigen. For example, WO2015/052538 describes a BCMA-specific CAR in which the binding domain is based on a proliferation-inducing ligand (APRIL), rather than a BCMA-binding antibody.

Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

Spacers commonly used in CAR design include an IgG1 Fc region, an IgG1 hinge or a CD8 stalk.

Transmembrane Domain

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:

(i) an ITAM-containing endodomain, such as the endodomain from CD3 zeta; and/or

(ii) a co-stimulatory domain, such as the endodomain from CD28 or ICOS; and/or

(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The CAR of the cell of the present invention may therefore comprise an antigen-binding component comprising an antigen-binding domain and a transmembrane domain; which is capable of interacting with a separate intracellular signalling component comprising a signalling domain. The vector of the invention may express a chimeric receptor signalling system comprising such an antigen-binding component and intracellular signalling component.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

Transgenic T-Cell Receptor (TCR)

The present invention provides an engineered cell which expresses a CAR or an engineered T-cell receptor (TCR).

The T-cell receptor (TCR) is a molecule found on the surface of T-cells which is responsible for recognizing fragments of antigen as peptides bound to major histocompatibility complex (MHC) molecules.

The TCR is a heterodimer composed of two different protein chains. In humans, in 95% of T-cells, the TCR consists of an alpha (α) chain and a beta (β) chain (encoded by TRA and TRB, respectively), whereas in 5% of T-cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example, the genes for engineered TCRs may be reintroduced into autologous T-cells and transferred back into patients for T-cell adoptive therapies. Such ‘heterologous’ TCRs may also be referred to herein as ‘transgenic TCRs’.

The transgenic TCR for use in the present invention may recognise a tumour associated antigen (TAA) when fragments of the antigen are complexed with major histocompatibility complex (MHC) molecules on the surface of another cell.

Target Antigen

A ‘target antigen’ is an entity which is specifically recognised by the CAR or TCR.

The target antigen may be an antigen present on a cancer cell, for example a tumour-associated antigen.

The target antigen for the CAR or TCR may be expressed at relatively low density on the target cell. Examples of tumour associated antigens which are known to be expressed at low densities in certain cancers include, but are not limited to, ROR1 in CLL, Typr-1 in melanoma, BCMA, and TACI in myeloma and ALK in Neuroblastoma.

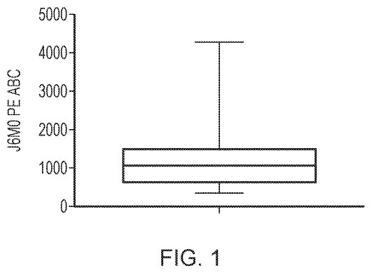

Example 1 describes a study investigating the expression of BCMA on myeloma cells. It was found that the range of BCMA copy number on a myeloma cell surface is low: at 348.7-4268.4 BCMA copies per cell with a mean of 1181 and a median of 1084.9 (FIG. 1).

The mean copy number of the target antigen for the CAR may be fewer than about 10,000; 5,000; 3,000; 2,000; 1,000; or 500 copies per target cell.

The copy number of an antigen on a cell, such as a cancer cell may be measured using standard techniques, such as using PE Quantibrite beads as described in Example 1.

The target antigen for the CAR may be expressed by the target cell at an average copy number of 1500 copies per cell or fewer, or 1000 copies per cell or fewer.

The target antigen may, for example, be BCMA.

BCMA

The B cell maturation target, also known as BCMA; TR17_HUMAN, TNFRSF17 (UniProt Q02223) is a transmembrane protein that is expressed in mature lymphocytes, e.g., memory B cells, plasmablasts and bone marrow plasma cells. BCMA is also expressed on myeloma cells. BCMA is a non-glycosylated type III transmembrane protein, which is involved in B cell maturation, growth and survival.

An antigen binding domain of a CAR which binds to BCMA may be any domain which is capable of binding BCMA.

As mentioned above, WO2015/052538 describes a BCMA-specific CAR in which the binding domain is based on a proliferation-inducing ligand (APRIL), rather than a BCMA-binding antibody. Carpenter et al (2013) describe an scFv-based anti-BCMA CAR.

The target antigen may be cleaved from the surface of a tumour cell by an ectoenzyme.

Agent

The present invention provides a cell which secretes an agent which blocks or reduces the activity of an ectoenzyme.

Examples of secreted agents suitable for use in the present invention include, but are not limited to, antibodies and antibody fragments, a single-domain antibodies (dAbs), diabodies and a non-antibody scaffold polypeptides.

An “antibody” (Ab) is a glycoprotein immunoglobulin which binds specifically to an antigen. An IgG antibody comprises two heavy (H) chains and two light (L) chains interconnected by disulfide bonds. Each H chain comprises a heavy chain variable region (VH) and a heavy chain constant region. The heavy chain constant region comprises three constant domains, CHI, CH2 and CH3. Each light chain comprises a light chain variable region (VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen.

Antibodies can include, for example, monoclonal antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (e.g. “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), minibodies, domain antibodies, synthetic antibodies (sometimes referred to herein as “antibody mimetics”), and peptibodies (i.e., Fc fusion molecules comprising peptide binding domains).

The agent may be bivalent (or multi-valent) and target two (or a plurality of) separate enzymatic domains on the ectoenzyme.

The secreted agent may have a molecular weight of less than about 100 kDa, less than about 50 kDa, less than about 25 kDa or less than about 20 kDa. The secreted agent may have a molecular weight of about 6 kDa to about 20 kDa or about 10 kDa to about 15 kDa.

The secreted agent may not require the formation of a disulfide bond for functional folding. As used herein “functional folding” may refer to the folding of the agent into a conformation in which it is capable of specifically binding to the ectoenzyme. “Functional folding” may refer to the folding of the agent into a conformation in which it is soluble and/or stable in the extracellular environment. In other words, the agent may comprise a disulfide bond, but the formation of this disulphide bond is not required for functional folding. The agent may not comprise a disulfide bond.

Single-Domain Antibodies

The secreted agent may be or may comprise a single-domain antibody (dAb).

dAbs are unique IgG molecules that are found naturally in for example camelids. Unlike conventional IgGs, dAbs are devoid of the light chain and lack the first constant domain of the heavy chain. Consequently, the antigen-binding fragment of dAbs is solely composed of a single variable domain, often referred to as a VHH. Cartilaginous fish also have heavy-chain antibodies (IgNAR, ‘immunoglobulin new antigen receptor’) from which single-domain antibodies called VNAR (variable new antigen receptor) fragments can be obtained.

dAbs are endowed with favorable characteristics such as size, solubility and affinity. In addition, due to the lack of intra chain disulfide bonds, these antibody fragments have been shown to be capable of productive folding in the reducing intracellular environment. As described above, these properties make dAbs suitable for targeting proteins within the cell.

dAbs have a molecular weight of about 12 to about 15 kDa and are typically about 110 amino acids in length.

The agent may be or comprise a VHH or VNAR.

By way of example, a dAb can be obtained by immunization of e.g. dromedaries, camels, llamas, alpacas or sharks with the desired antigen and subsequent isolation of the mRNA coding for heavy-chain antibodies. Reverse transcription and PCR can then be used to generate a library of dAbs. Standard screening techniques such as phage display and ribosome display may be used to identify the suitable clones binding the antigen of interest.

Once the most potent clones have been identified, their DNA sequence may optimized, for example to improve their stability towards enzymes. Humanization may also be performed.

dAbs may be expressed in a cell using conventional vectors, such as those described herein.

The agent may be a bivalent dAb, or diabody, in which two dAb are joined using a flexible linker, such as a serine-glycine linker. The two dAbs may target separate enzymatic domains on the same ectoenzyme target antigen.

Non-Antibody Scaffolds

The secreted agent may be or comprise a non-antibody scaffold. As used herein, non-antibody scaffold refers to a binding polypeptide that does not bind to its target polypeptide via complementary determining regions (CDRs).

The non-antibody scaffold may be a domain-sized scaffold. In particular, the non-antibody scaffold may be a domain-sized scaffold with a molecular weight from about 6 kDa to about 20 kDa.

Non-antibody scaffolds bind to a target polypeptide via a range of different polypeptide conformational architectures which mediate protein-protein interactions.

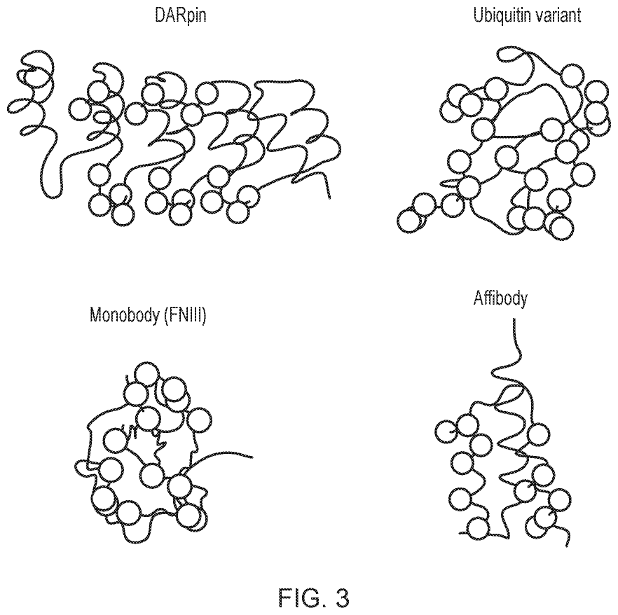

A summary of suitable, illustrative non-antibody scaffolds is shown in Table 1 and FIG. 3.

TABLE 1

| | | | | | Structural |
|---|---|---|---|---|---|
| | Parental | | Specificity | MW | disulfide |
| Scaffold | protein | Structure | Derivation | kDa | bond(s) |
| Affibodies | Z domain | α-helical | Helix | 6 | No |
| | | | randomization | | |
| Affilins | Ubiquitin | α/β | Beta strand | 10 | No |
| | | | randomization | | |
| Anticalins | Lipocalin | B Sheet α | Loop | 20 | No |
| | | helix | randomization | | |
| | | terminus | | | |
| Atrimers | C-type | α/β | Loop | 3 × 20 | No |
| | Lectin | | randomization | | |
| DARPins | Ankyrin | α helix + β | Helix | 14-21 | No |
| | repeats | turn | randomization | | |
| | | | β turn | | |
| | | | randomization | | |
| FN3 | Fibronectin | β Sheet | Loop | 10 | No |
| scaffolds | (type III) | | randomization | | |
| (Adnectins, | | | B strand | | |
| Centyrins, | | | randomization | | |
| Pronectins, | | | | | |
| Tn3) | | | | | |
| Fynomers | SH3 domain | β Sheet | Loop | 7 | No |
| | (fyn kinase) | | randomization | | |
| Kunitz | Serine | α/β | Loop | 7 | Yes |
| domains | protease | | randomization | | |
| | inhibitors | | | | |
| O-bodies | OB-fold | β Sheet | Loop | 12 | No |
| | | | randomization | | |
| | | | Beta sheet | | |
| | | | randomization | | |

The non-antibody scaffold domain may comprise an fibronectin type III (FN3) scaffold (e.g. Adnectins and Centyrins), Fynomer, Affibody, Affilin, Anticalin, Atrimer, DARPin, Pronectin or O-Body.

The non-antibody scaffold domain may comprise a FN3 scaffold.

FN3 scaffolds may be generated from combinatorial libraries in which portions of the FN3 scaffold are diversified using molecular display and directed evolution technologies such as phage display, mRNA display and yeast surface display. A large number of FN3 scaffolds that have high affinity and high specificity to their respective targets are known in the art. FN3 scaffolds have a structure similar to antibody variable domains, with seven beta sheets (referred to as A-G) forming a beta-sandwich and three exposed loops on each side corresponding to the three complementarity determining regions. By way of example, there are two distinct designs of FN3 libraries that have been successful. The first type modifies some or all of the loops BC (between the second and third beta sheets), DE (between the fourth and fifth beta sheets) and FG (between the sixth and seventh sheets). This design creates diversified positions on a convex surface that is suitable for targeting concave surfaces such as enzyme active sites. The second type modifies positions in some or all of the C, D, F and G (or the 3rd, 4th, 6th and 7th) strands in addition to the CD and FG loops. This design creates a flatter, slightly concave surface that is suitable for targeting surfaces typically involved in protein-protein interactions.

The non-antibody scaffold domain may comprise a Fynomer scaffold.

Ectoenzyme

The agent secreted by the cell of the present invention is capable of blocking or reducing the activity of an ectoenzyme. The agent may specifically bind to one or more enzymatic sites on the ectoenzyme.

An ectoenzyme is an enzyme which functions outside of a cell. It may be secreted by a cell or expressed on the outer surface of a cell. Enzymes which are secreted by a cell and function outside of the cell are also known as exoenzymes or extracellular enzymes.

Ectoenzymes are involves in many biological processes, particularly in the breakdown of macromolecules.

The ectoenzyme blocked by secreted agent of the present invention may be involved in the production or metabolism of a factor in the microenvironment which is either
(i) required by a tumour cell for survival, proliferation, metastasis or chemoresistance, or
(ii) detrimental to the survival, proliferation or activity of the CAR- or TCR-expressing cell.

The ectoenzyme may be secreted by a tumour cell or expressed on the surface of a tumour cell.

The ectoenzyme may be enolase 1 (ENO1). ENO1 is expressed on the tumour cell surface during pathological conditions such as inflammation, autoimmunity, and malignancy. ENO1 overexpression has been associated with multiple tumours, including glioma, neuroendocrine tumours, neuroblastoma, pancreatic cancer, prostate cancer, cholangiocarcinoma, thyroid carcinoma, lung cancer, hepatocellular carcinoma, and breast cancer. ENO1 promotes cell proliferation by regulating the PI3K/AKT signalling pathway and induces tumorigenesis by activating plasminogen. Its role as a plasminogen receptor leads to extracellular matrix degradation and cancer invasion. ENO1 inhibition reduces suppression by dendritic cells.

The ectoenzyme may be an immunosuppressive enzyme. It may modify the nutrient availability and/or lead to the production of toxic catabolites in the tumour microenvironment.

Amino Acid Metabolism

Enzymes that catabolize amino acids are frequently overexpressed or ectopically expressed in cancerous tissues. The ectoenzyme blocked by secreted agent of the present invention may be involved in the catabolism of one or more amino acids, such as arginine, tryptophan or phenylalanine. In this respect, the ectoenzyme may be selected from one of the following: inducible nitric oxide synthase (iNOS), type 1 and type 2 arginases (Arg1 and Arg2), type 1 and type 2 indoleamine 2,3-dioxygenases (IDO1 and IDO2), tryptophan 2,3 dioxygenase (TDO), and interleukin 4 (IL-4)-induced gene 1 (IL4I1).

Ectonucleotidases

The exoenzyme may be an ectonucleotidase which metabolise nucleotides. These enzymes are commonly expressed on the plasma membrane and have externally oriented active sites.

The purinergic system represents a second mechanism for the fine regulation of the immune response. The purinergic mediators, ATP and adenosine, are mostly intracellular and are released following stress-induced stimuli. ATP accumulates outside the cell by lytic processes induced by necrosis, as well as active processes, such as exocytosis of vesicles containing the nucleotide or passage through purinergic channels. Certain ectoenzymes dephosphorylate extracellular ATP to adenosine and are known to significantly contribute to purinergic halo. The accumulation of adenosine results in a simultaneous decrease in the immunostimulatory and increase in the immunosuppressive signal.

The ectoenzyme blocked by secreted agent of the present invention may be involved in the degradation of nucleotide adenosine triphosphate (ATP) to adenosine. For example, the ectoenzyme may be selected from: CD39 and CD73 which are expressed on the cell surface. CD39 (also known as ENTPD1) is involved in the first step in the production of adenosine involving the conversion of ATP/ADP to AMP. CD73 (also known as NT5E) is involved in the second step involving the conversion of AMP to adenosine.

Cleavage of Transmembrane Protein

The ectoenzyme blocked by secreted agent of the present invention may have the capacity to cleave a transmembrane protein. The ectoenzyme may cleave the target antigen for the CAR or TCR of the cell of the present invention.

Blocking the action of such an enzyme may enhance CAR- or TCR-mediated activity for two reasons: a) it increases the effective concentration of the target antigen at the target cell surface, as the portion of the target antigen containing the epitope for the CAR or TCR is not removed by cleavage; and b) it decreases the amount of soluble antigen in the tumour microenvironment which competes with the membrane-bound antigen for binding to the CAR.

Gamma Secretase

The ectoenzyme may, for example, be gamma secretase, a multi-subunit protease complex, itself an integral membrane protein, that cleaves single-pass transmembrane proteins at residues within the transmembrane domain.

The gamma secretase complex consists of four individual proteins: PSEN1 (presenilin-1), nicastrin, APH-1 (anterior pharynx-defective 1), and PEN-2 (presenilin enhancer 2). A fifth protein, known as CD147, may be a non-essential regulator of the complex whose absence increases activity. Presenilin, an aspartyl protease, is the catalytic subunit; and modultes immune cell activity. Nicastrin's primary role is in maintaining the stability of the assembled complex and regulating intracellular protein trafficking. PEN-2 associates with the complex via binding of a transmembrane domain of presenilin and, among other possible roles, helps to stabilize the complex after presenilin proteolysis has generated the activated N-terminal and C-terminal fragments. APH-1, which is required for proteolytic activity, binds to the complex via a conserved alpha helix interaction motif and aids in initiating assembly of premature components.

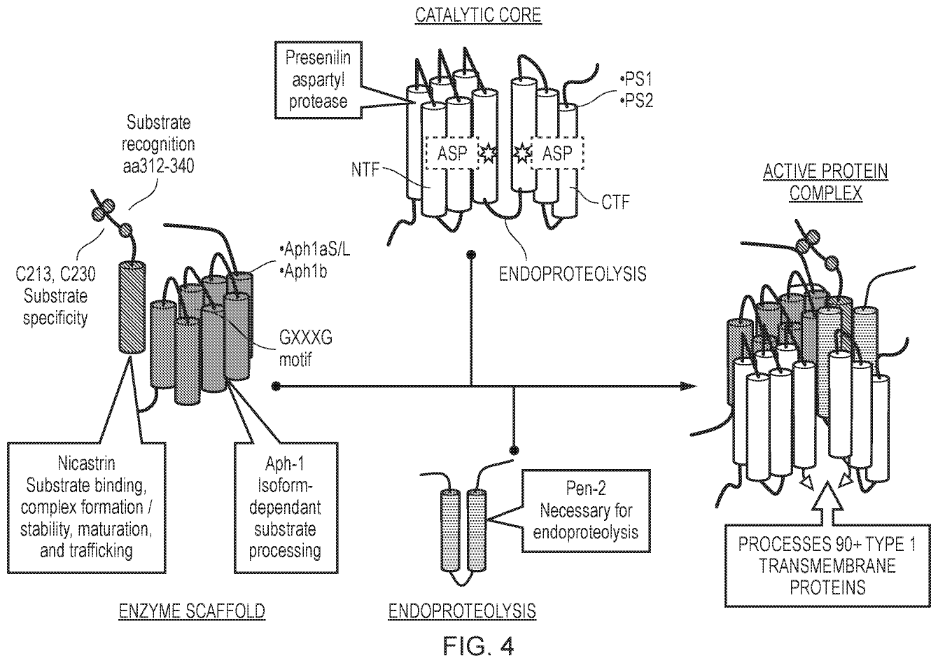

The gamma secretase complex is formed by the sequential assembly of APH1, nicastrin, presenilin (PS), and PEN-2, as illustrated schematically in FIG. 4. The four components cross-regulate each other coordinately.

Gamma secretase has many substrates, including notch receptors, β-amyloid precursor protein (APP) and B-cell maturation antigen (BCMA). Interaction of the gamma secretase complex with the γ-secretase activating protein facilitates the gamma cleavage of amyloid precursor protein into β-amyloid. Substrate recognition occurs via nicastrin ectodomain binding to the N-terminus of the target, which is then passed between the two presenilin fragments to a water-containing active site where the catalytic aspartate residue is located.

The agent secreted by the cell of the present invention may bind one of the components of the gamma secretase complex. In particular, it may bind nicastrin or presenilin.

The amino acid sequences of the gamma secretase complex are publicly available as summarised in Table 2 and the amino acid sequences for nicastrin and presenilin are shown below as SEQ ID No. 1 and 2 respectively.

TABLE 2
components of the gamma secretase complex

| Protein | Uniprot accession No. |
|---|---|
| nicastrin | Q92542 |
| presenilin | P49768 |
| APH-1 | Q8WW43 |
| PEN-2 | Q9NZ42 |

SEQ ID No. 1 (nicastrin)
MATAGGGSGADPGSRGLLRLLSFCVLLAGLCRGNSVERKIYIPLNKTAPC
VRLLNATHQIGCQSSISGDTGVIHVVEKEEDLQWVLTDGPNPPYMVLLES
KHFTRDLMEKLKGRTSRIAGLAVSLTKPSPASGFSPSVQCPNDGFGVYSN
SYGPEFAHCREIQWNSLGNGLAYEDFSFPIFLLEDENETKVIKQCYQDHN
LSQNGSAPTFPLCAMQLFSHMHAVISTATCMRRSSIQSTFSINPEIVCDP
LSDYNVWSMLKPINTTGTLKPDDRVVVAATRLDSRSFFWNVAPGAESAVA
SFVTQLAAAEALQKAPDVTTLPRNVMFVFFQGETFDYIGSSRMVYDMEKG
KFPVQLENVDSFVELGQVALRTSLELWMHTDPVSQKNESVRNQVEDLLAT
LEKSGAGVPAVILRRPNQSQPLPPSSLQRFLRARNISGVVLADHSGAFHN
KYYQSIYDTAENINVSYPEWLSPEEDLNFVTDTAKALADVATVLGRALYE
LAGGTNFSDTVQADPQTVTRLLYGFLIKANNSWFQSILRQDLRSYLGDGP
LQHYIAVSSPTNTTYVVQYALANLTGTVVNLTREQCQDPSKVPSENKDLY
EYSWVQGPLHSNETDRLPRCVRSTARLARALSPAFELSQWSSTEYSTWTE
SRWKDIRARIFLIASKELELITLTVGFGILIFSLIVTYCINAKADVLFIA
PREPGAVSY

The agent secreted by the cell of the present invention may bind nicastrin. In particular, it may bind to an epitope within the region of nicastrin from amino acids 333-393. This portion of the sequence is shown in bold and underlined in SEQ ID No. 1 above.

SEQ ID No. 2 (presenilin)
MTELPAPLSYFQNAQMSEDNHLSNTVRSQNDNRERQEHNDRRSLGHPEPL
SNGRPQGNSRQVVEQDEEEDEELTLKYGAKHVIMLFVPVTLCMVVVVATI
KSVSFYTRKDGQLIYTPFTEDTETVGQRALHSILNAAIMISVIVVMTILL
VVLYKYRCYKVIHAWLIISSLLLLFFFSFIYLGEVFKTYNVAVDYITVAL
LIWNFGVVGMISIHWKGPLRLQQAYLIMISALMALVFIKYLPEWTAWLIL
AVISVYDLVAVLCPKGPLRMLVETAQERNETLFPALIYSSTMVWLVNMAE
GDPEAQRRVSKNSKYNAESTERESQDTVAENDDGGFSEEWEAQRDSHLGP
HRSTPESRAAVQELSSSILAGEDPEERGVKLGLGDFIFYSVLVGKASATA
SGDWNTTIACFVAILIGLCLTLLLLAIFKKALPALPISITFGLVFYFATD
YLVQPFMDQLAFHQFYI

The agent secreted by the cell of the present invention may bind presenilin. In particular, it may bind to an epitope within the region of presenilin from amino acids 101-139. This portion of the sequence is shown in bold and underlined in SEQ ID No. 2 above. In particular, the antibody may target residues K101 and or S102 of presenilin.

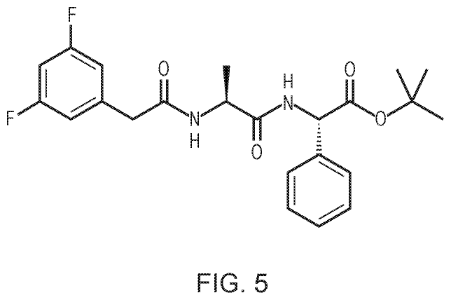

The agent may be a small molecule gamma secretase inhibitor such as DAPT (tert-Butyl (2S)-2-[[(2S)-2-[[2-(3,5-difluorophenyl)acetyl]amino]propanoyl]amino]-2-phenylacetate), which has the structure shown in FIG. 5.

Alternatively, the agent may be an antibody-like binder, such as an scFv or a dAb.

Various gamma-secretase binding antibodies/scFvs have been previously described, such as those summarised in Table 3. The cell of the present invention may secrete one of these antibodies or scFvs or a derivative thereof.

TABLE 3

| Antibody/scFv | Binds | Reference |
|---|---|---|
| G9 | A conformational epitope in an | Zhang et al 2014 J Biol Chem |
| | amino-terminal segment of | 289:34851-34861 |
| | nicastrin contained within the | |
| | region encoded by exons 1-6 | |
| A5201F | The extracellular domain of | Hayashi et al 2009. J Biol. |
| | Nicastrin | Chem. 284:27838-27847. |
| | | Note: the VH and VL |
| | | sequences of A5201F are |
| | | shown in FIG. 1A. |
| A5226A | The extracellular domain of | Hayashi et al 2012. |
| | Nicastrin (mature form) | Oncogene 31:787-798 |
| 9D11 | Presenilin. Transmembrane | Takagi-Niidome et al 2013. |
| | domains (TMDs) 1, 6, 7, and 9 | Biochemistry 52:61-9 |
| | of presenilin form the “catalytic | |
| | pore” structure within the | |
| | membrane for intramembrane | |
| | proteolysis. 9D11, binds TMD1- | |
| | proximal residues in the | |
| | hydrophilic loop region. | |

Examples 5 and 6 describe the generation of presenilin- and nicastrin-specific antibodies respectively.

The VH and VL sequences for three gamma-secretase binding antibodies are given below as SEQ ID Nos. 3-8 with CDR sequences shown in bold and underlined. The cell of the present invention may secrete an antibody or fragment thereof (such as an scFv) comprising the CDRs from any of these antibodies.

SEQ ID No. 3 (10C5 VH)
QVTLKESGPGMLQPSKTLSLTCSFSGFSLRSSGMVVSWIRQPSGKSLEWL
AAIDWDGDKYYNPSLKSRLTVSKDTSNTQVFLKITSVDIADTATYYCARI
YYGYYFDYWGQGVMVTVSS
SEQ ID No. 4 (10C5 VL)
DIVMTQSPSSLVVSAGETVTINCKSSQSLLSSGNQKNYLAWYQQKPGQSP
KLLIYWASTRQSGVPDRFIGSGSGTDFTLTISSVQAEDLAIYYCQQYFEN
LPWTFGGGTKLELKR
SEQ ID No. 5 (1E2 VH)
EVQLVESGGGLVQPGRSMKLSCAASGFTFSNYYMAWVRQAPTKGLEWVAS
ISTGGGNTYYRDSVKGRFTISRDNAKSTLYLQMDSLRSEDTATYYCARHR
FGVPHYFDYWGQGVMVTVSS
SEQ ID No. 6 (1E2 VL)
DIVMTQTPSSQAVSAGEKVTMSCKSSQSLLYSENKKNYLAWYQQKPGQSP
KLLIYWASTRESGVPDRFIGSGSGTDFTLTISSVQAEDLAVYYCQQYYNF
NTFGAGTKLELKR
SEQ ID No. 7(10C11 VH)
QVQLKESGPGLVQPSETLSLTCTVSGFSLTSYSVSWVRQPSGKGPEWMGR
MWYDGDTAYNSALKSRLSISRDTSKNQVFLKMNSLQTDDTGTYYCTRDPW
DWGQGVMVTVSS
SEQ ID No. 8 (10C11 VL)
DVVLTQTPSTLSATIGQSASISCRSSQSLLDSSGNTFLYWYLQRPGQSPQ
LLIYLVSILGSGVPNRFSGSGSGTDFTLKISGVEAEDLGVYYCMQATHAP
LTFGSGTKLEIKR

The gamma secretase-binding antibody or fragment thereof may comprise the VH and/or VL sequence from 1005, 1E2 or 10C11, as described above, or a variant thereof which has at least 70, 80, 90 or 90% sequence identity, which variant retains the capacity to bind gamma secretase.

The present invention also provides a new gamma secretase-binding antibody or antibody fragment, comprising a) a heavy chain variable region (VH) having complementarity determining regions (CDRs) as shown in SEQ ID No. 3, and b) b) a light chain variable region (VL) having complementarity determining regions (CDRs) as shown in SEQ ID No. 4.

The antibody or antibody fragment may comprise a VH region comprising the sequence shown in SEQ ID No. 3 and a VL region comprising the sequence shown in SEQ ID No. 4.

The present invention also provides a new gamma secretase-binding antibody or antibody fragment, comprising a) a heavy chain variable region (VH) having complementarity determining regions (CDRs) as shown in SEQ ID No. 5, and b) b) a light chain variable region (VL) having complementarity determining regions (CDRs) as shown in SEQ ID No. 6.

The antibody or antibody fragment may comprise a VH region comprising the sequence shown in SEQ ID No. 5 and a VL region comprising the sequence shown in SEQ ID No. 6.

The present invention also provides a new gamma secretase-binding antibody or antibody fragment, comprising a) a heavy chain variable region (VH) having complementarity determining regions (CDRs) as shown in SEQ ID No. 7, and b) b) a light chain variable region (VL) having complementarity determining regions (CDRs) as shown in SEQ ID No. 8.

The antibody or antibody fragment may comprise a VH region comprising the sequence shown in SEQ ID No. 7 and a VL region comprising the sequence shown in SEQ ID No. 8.

The invention also provides a nucleic acid encoding such an or antibody fragment, a vector comprising such a nucleic acid and a cell which comprises such a nucleic acid and secretes the antibody or antibody fragment.

Nucleic Acid Construct

The present invention also provides a nucleic acid construct which comprises: a first polynucleotide which encodes a chimeric antigen receptor (CAR) or engineered transgenic T-cell receptor (TCR); and a second polynucleotide which encodes an agent which blocks or reduces the activity of an ectoenzyme.

The first and second polynucleotides may be in either order on the construct.

The present invention also provides a kit of polynucleotides which comprises: a first polynucleotide which encodes a chimeric antigen receptor (CAR) or engineered transgenic T-cell receptor (TCR); and a second polynucleotide which encodes an agent which blocks or reduces the activity of an ectoenzyme.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid construct may also comprise a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No. 9 (RAEGRGSLLTCGDVEENPGP).

Vector

The present invention also provides a vector, or kit of vectors, which comprises one or more nucleic acid sequence(s) encoding a chimeric antigen receptor (CAR) or engineered transgenic T-cell receptor (TCR); and/or an agent which blocks or reduces the activity of an ectoenzyme. Such a vector may be used to introduce the nucleic acid sequence(s) into a host cell so that it expresses a CAR/TCR and/or secretes an agent.

The vector may, for example, be a plasmid or a viral vector, such as a retroviral vector or a lentiviral vector, or a transposon based vector or synthetic mRNA.

The vector may be capable of transfecting or transducing a cell, such as a T cell or a NK cell.

Cell

The present invention provides a cell which expresses a chimeric antigen receptor (CAR) or engineered T-cell receptor (TCR) and secretes an agent which blocks or reduces the activity of an ectoenzyme.

The cell may comprise a nucleic acid sequence, construct or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+ FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells according to the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo cell from a subject. The cell may be from a peripheral blood mononuclear cell (PBMC) sample. The cells may be activated and/or expanded prior to being transduced with nucleic acid encoding the molecules providing the chimeric polypeptide according to the first aspect of the invention, for example by treatment with an anti-CD3 monoclonal antibody.

The cell of the invention may be made by:
(i) isolation of a cell-containing sample from a subject or other sources listed above; and
(ii) transduction or transfection of the cells with one or more a nucleic acid sequence(s) encoding a chimeric polypeptide.

The cells may then by purified, for example, selected on the basis of expression of the antigen-binding domain of the antigen-binding polypeptide.

Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The cell-containing sample may be isolated from a subject or from other sources, as described above.

The present invention provides a cell of the present invention for use in treating and/or preventing a disease.

The invention also relates to the use of a cell of the present invention in the manufacture of a medicament for the treatment of a disease.

The disease to be treated by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The disease may be a plasma cell disorder such as plasmacytoma, plasma cell leukemia, multiple myeloma, macroglobulinemia, amyloidosis, Waldenstrom's macroglobulinemia, solitary bone plasmacytoma, extramedullary plasmacytoma, osteosclerotic myeloma, heavy chain diseases, monoclonal gammopathy of undetermined significance or smoldering multiple myeloma.

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

The invention will now be further described by way of Examples, which are meant to serve to assist one of ordinary skill in the art in carrying out the invention and are not intended in any way to limit the scope of the invention.

### EXAMPLES

### Example 1—Expression of BCMA on Surface of Multiple Myeloma Cells

Primary myeloma cells were isolated by performing a CD138 immunomagnetic selection on fresh bone marrow samples from Multiple myeloma patients that were known to have frank disease. These cells were stained with the BCMA specific J6MO mAb (GSK) which was conjugated to PE. At the same time, a standard of beads with known numbers of binding sites was generated using the PE Quantibrite bead kit (Becton Dickenson) as per the manufacturer's instructions. The BCMA copy number on myeloma cells was derived by correlating the mean-fluorescent intensity from the myeloma cells with the standard curve derived from the beads. It was found that the range of BCMA copy number on a myeloma cell surface is low: at 348.7-4268.4 BCMA copies per cell with a mean of 1181 and a median of 1084.9 (FIG. 1). This is considerably lower than e.g. CD19 and GD2, classic targets for CARs.

### Example 2—Generation of Gamma Secretase Targeting DAbs

Llama vaccination is used to generate single domain binders to gamma secretase (GST). Animals are vaccinated with peptides consisting of key residues on presenilin (PS1) and nicastrin (GST complex components) conjugated to an immunogen. Hybridomas are generated and screened on recombinant protein, blocking activity is assessed on a GST dependent cell line (A549) and affinity is assessed by surface plasmon resonance. Bivalent DAbs are created using flexible serine-glycine linkers to conjugate single domain inhibitory binders that target separate enzymatic domains.

### Example 3—Assessing Ectoenzyme Inhibition by Anti-GST DAbs

The dAbs generated in Example 2 are purified and incubated with MM cell lines (expressing low/high BCMA) and primary bone marrow (BM) derived MM cells in vitro. Changes to BCMA expression or sBCMA are quantified by FACs and ELISA respectively and compared to DAPT (a GST blocking molecule). GST blockade (via notch) can affect T cell function so the effect of DAb on resident and CAR T cells is assessed by CD3/CD28 mediated proliferation and cytokine release following GST blockade. If there is a significant deficit to T cell function, CAR T cells are cotransduced to express a PS1 mutant and anti nicastrin DAb to maintain GST function on the T cell as PS1 mutations have been associated with GST function independent of nicastrin.

### Example 4—Investigating the Effect of GST Inhibition on CAR Function

T cells are transduced with a construct which co-expresses a dAb identified in Example 2 with an anti-BCMA CAR comprising truncated APRIL as the antigen-binding domain, as described in WO2015/052538. The effect on tumour killing is assessed, in comparison to T cells expressing the APRIL CAR alone, in vitro and using established in vivo models.

### Example 5—Generation of Presenilin-Specific Antibodies

A peptide consisting of residues K101-M139 of presenilin was fused to the immunogen keyhole limpet haemocyanin (KLH). Three Wistar rats were immunised with the KLH pSEN1 peptide.

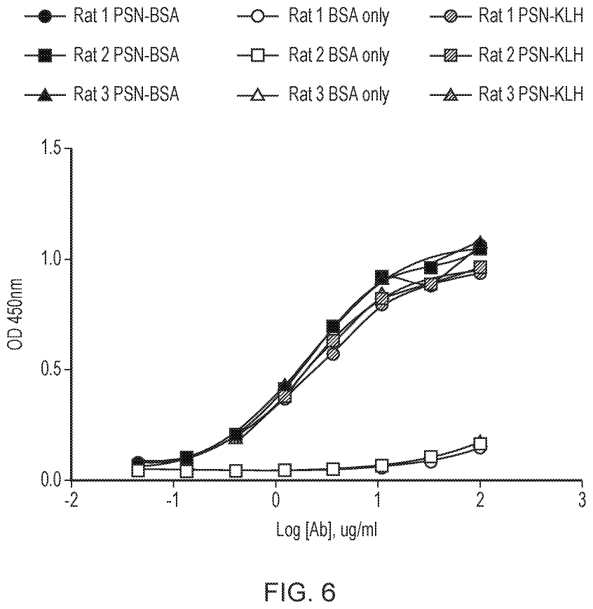

In order to select for sera reactive against the pSEN1 peptides, rather than KLH, the PSEN1 peptide was conjugated to maleimide-BSA via cysteine at its N terminus, and sera from the immunised rats was tested against BSA PSEN1 peptide and BSA CD79 peptide (negative control). The data are shown in FIG. 6.

Sera were tested by ELISA for binding to PSEN1-BSA, PSEN1-KLH or BSA only (i.e. the BSA CD79 control) at a starting dilution of 1 in 100, followed by 1 in 3 serial dilution. All three rats showed positive seroconversion so lymphocytes from all three rats were fused with myeloma cells to form hybridomas.

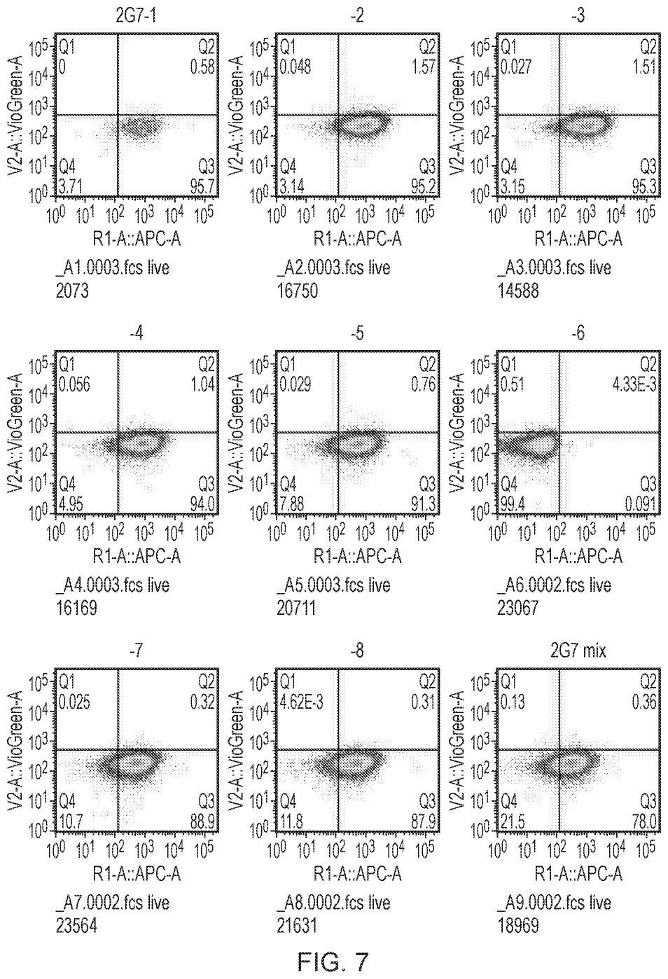

Of the 54 clones screened, one tested positive for BSA PSEN1 peptide binding (data not shown). This clone (2G7) was selected to be expanded and single cloned in order to select for the monoclonal antibody from the hybridoma clone that bound to A549 cells. A549 cells are derived from a non-small lung cancer and are highly sensitive to γ-secretase inhibitors. The data are shown in FIG. 7. The single clones 2G7-1, -2, -3, -4, -5, -7 and -8 showed positive binding on A549, as well as positive control anti-Nicastrin. The antibodies from those clones were sequenced, expressed in a vector and expressed on CHO cells for characterization.

### Example 6—Generation of Nicastrin-Specific Antibodies

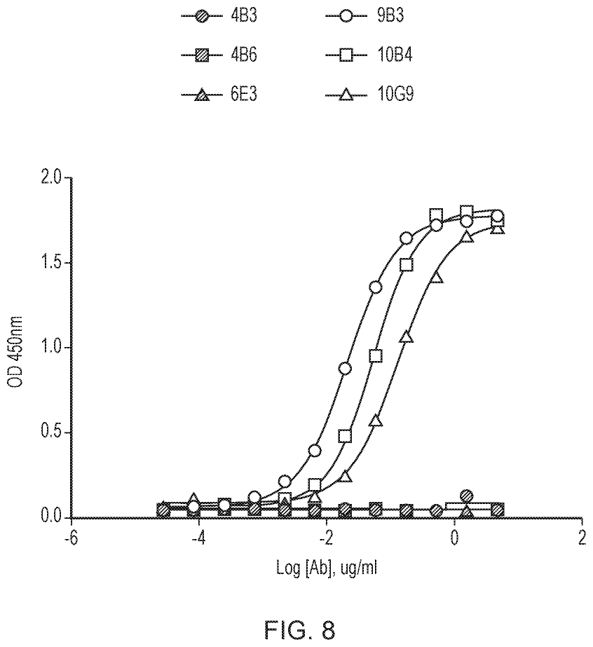

Three Wistar rats were immunised with human nicastrin. All three rats showed positive seroconversion, so lymphocytes from all 3 rats were taken and fused to myeloma cells to generate hybridoma clones. Of the 54 clones tested, six clones tested positive on cells, as shown in FIG. 8. These clones were then screened on HEK cells expressing chimeric Nicastrin to select for binders that targeted a conserved region from amino acid 333-393 of nicastrin.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which expresses a chimeric antigen receptor (CAR) or engineered T-cell receptor (TCR) and secretes an agent which blocks or reduces the activity of an ectoenzyme.

2. A cell according to claim 1 wherein the agent comprises an antibody or fragment thereof, a single-domain antibody, a diabody or a non-antibody scaffold polypeptide.

3. A cell according to claim 1, where-in the agent is bivalent and targets two enzymatic domains on the ectoenzyme.

4. A cell according to claim 1, wherein the ectoenzyme is secreted by or expressed on the outer surface of a tumour cell.

5. A cell according to claim 1, wherein blocking or reducing the activity of the ectoenzyme directly or indirectly affects the target antigen for the CAR or engineered TCR.

6. A cell according to claim 5, wherein, in the absence of the agent, the ectoenzyme cleaves the target antigen from the target cell surface.

7. A cell according to claim 5, wherein blocking or reducing the activity of the ectoenzyme increases the level of target antigen on the target cell.

8. A cell according to claim 1, wherein ectoenzyme activity cleaves a transmembrane protein.

9. A cell according to claim, wherein the target antigen for the CAR or engineered TCR is B cell maturation antigen (BCMA) and the ectoenzyme is gamma secretase (GST).

10. A cell according to claim 1, wherein blocking or reducing the activity of the ectoenzyme directly or indirectly reduces immune suppressing factors.

11. A cell according to claim 10, wherein the immune suppressing factor is adenosine and the ectoenzyme is selected from one of the following ectonucleotidases: CD39 and CD73.

12. A cell according to claim 1, wherein blocking or reducing the activity of the ectoenzyme directly or indirectly reduces an immune suppressing cell type.

13. A cell according to claim 12, wherein the immune suppressing cell type is dendritic cells and the ectoenzyme is the glycolytic enzyme ENO1.

14. A nucleic acid construct which comprises: a first polynucleotide which encodes a chimeric antigen receptor (CAR) or engineered transgenic T-cell receptor (TCR); and a second polynucleotide which encodes an agent which blocks or reduces the activity of an ectoenzyme.

15. A nucleic acid construct according to claim 14 wherein the first and second polynucleotides are separated by a co-expression site.

16. A vector comprising a nucleic acid construct according to claim 14.

17-18. (canceled)

19. A pharmaceutical composition which comprises a plurality of cells according to claim 1.

20. (canceled)

21. A method for treating cancer, which comprises the step of administering a pharmaceutical composition according to claim 19 to a subject in need thereof.

22. A method according to claim 21, which comprises the following steps:
(i) isolating a cell containing sample;
(ii) introducing a nucleic acid construct into the cells ex vivo, wherein the nucleic acid construct nucleic acid construct comprises: a first polynucleotide which encodes a chimeric antigen receptor (CAR) or engineered transgenic T-cell receptor (TCR); and a second polynucleotide which encodes an agent which blocks or reduces the activity of an ectoenzyme; and
(iii) administering the cells from (ii) to a subject.

23. (canceled)

24. A method for making a cell according to claim 1, which comprises the step of introducing a first polynucleotide which encodes a chimeric antigen receptor (CAR) or engineered transgenic T-cell receptor (TCR); and a second polynucleotide which encodes an agent which blocks or reduces the activity of an ectoenzyme into a cell ex vivo.
